(19) United States
(12) Patent Application Publication
(10) Pub. No.: US 2023/0115209 A1
(43) Pub. Date: Apr. 13, 2023

(54) PSILOCYBIN AND PSILOCIN CONTAINING COMPOSITIONS AND METHODS OF USING AND MAKING THE SAME

(71) Applicant: Orthogonal Thinker, Inc., Kihei, HI (US)

(72) Inventor: Chester Herbert Watson, Missoula, MT (US)

(21) Appl. No.: 17/912,821

(22) PCT Filed: Mar. 19, 2021
(86) PCT No.: PCT/US2021/023103

§ 371 Date: Sep. 19, 2022

Related U.S. Application Data
(60) Provisional application No. 62/992,263, filed on Mar. 20, 2020.

(51) Int. Cl. A61K 31/661; A61K 45/06; A61K 36/07; A61K 31/4045

(52) U.S. Cl. CPC A61K 31/661; A61K 45/06; A61K 36/07; A61K 31/4045; A61K 2236/17; A61K 2236/333; A61K 2236/51

(57) ABSTRACT

A composition is provided. The composition contains: (A) a psychoactive compound selected from the group consisting of psilocybin, psilocin, and combinations thereof; and (B) a supplement selected from the group consisting of an amino acid, a vitamin B6, piracetam, gamma aminobutyric acid (GABA), theobromine, caffeine, resveratrol, and combinations thereof. A method of producing the composition and an oral dosage containing the composition are also provided.

### PRIORITY CLAIM

The present disclosure claims priority under 35 U.S.C. 365 and U.S.C. 119(a) to PCT/US2021/023103 (“the PCT '103 Application”), filed Mar. 19, 2021, which claims priority under 35 U.S.C. 119(e) to U.S. provisional application 62/992,263, filed Mar. 20, 2020 (“the Provisional '263 Application”) The disclosures of the PCT '103 Application and the Provisional '263 Application are incorporated herein in their entireties.

### TECHNICAL FIELD

The present disclosure relates generally to compositions and methods of forming compositions that contain a psychoactive compound selected from the group consisting of psilocybin, psilocin, and combinations thereof.

### BACKGROUND

Medicinal uses of psilocybin and/or psilocin are currently being investigated for a number of functions, such as for enhancing cognitive, emotional and/or perceptual functions of an individual; or for promoting neurogenesis, resolving neuropathy, and/or improving neurological health of an individual. Continuing efforts are being made to develop medicinal compositions containing psilocybin and/or psilocin in combination with other components to enhance and/or add to the medicinal properties of the psilocybin and/or psilocin.

The art recognizes the need for compositions containing psilocybin and/or psilocin that are suitable for medicinal use.

### SUMMARY OF DISCLOSURE

According to the present disclosure, composition is provided. The composition contains: (A) a psychoactive compound selected from the group consisting of psilocybin, psilocin, and combinations thereof; and (B) a supplement selected from the group consisting of an amino acid, a vitamin B6, piracetam, gamma aminobutyric acid (GABA), theobromine, caffeine, resveratrol, and combinations thereof.

A method for making a composition is provided. The method includes (a) providing ethanol, distilled from a rye-containing mash; (b) providing dried and cured psilocybin-containing mushrooms, and grinding the mushrooms to obtain a particulate material; (c) soaking the particulate material in water; (d) adding distilled ethanol to the soaked particulate material, mixing to carry out ethanol extraction, and filtering to obtain an ethanol extract; (e) adding distilled ethanol to the ethanol extract to yield a solution; (f) stirring the solution; (g) heating the solution while stirring to cause evaporation, thereby obtaining a sludge; and (h) drying the sludge using vacuum to produce a powder material, thereby obtaining the composition.

A composition made by the method is also provided.

An oral dosage containing the composition is provided.

The disclosure further provides for methods of treatment by administering the compositions described herein. In one aspect, the method enhances cognitive, emotional and/or perceptual functions by administering any of the compositions described herein. In another aspect, the method promotes neurogenesis, resolving neuropathy, and/or improving neurological health by administering any of the compositions described herein.

### DETAILED DESCRIPTION

“Alkyl” and “alkyl group” refer to a saturated linear, cyclic, or branched hydrocarbon group. Nonlimiting examples of suitable alkyl groups include methyl, ethyl, n-propyl, i-propyl, n-butyl, t-butyl, i-butyl (or 2-methylpropyl), etc. In one embodiment, the alkyl group has from 1 to 20 carbon atoms.

“Alkenyl” or “alkenyl group” refer to a hydrocarbyl group containing at least one C═C double bond. Alkenyl groups may be linear, cyclic or branched. Nonlimiting examples of suitable alkenyl groups include ethenyl groups, n-propenyl groups, i-propenyl groups, n-butenyl groups, t- butenyl groups, i-butenyl groups, etc.

“Aralkyl” and “aralkyl group” refer to an organic radical derived from aromatic hydrocarbon by replacing one or more hydrogen atoms with an aryl group.

“Aryl” and “aryl group” refer to an organic radical derived from aromatic hydrocarbon by deleting one hydrogen atom therefrom. An aryl group may be a monocyclic and/or fused ring system, each ring of which suitably contains from 5 to 7, or from 5 or 6 atoms. Structures wherein two or more aryl groups are combined through single bond(s) are also included. Specific examples include, but are not limited to, phenyl, tolyl, naphthyl, biphenyl, anthryl, indenyl, fluorenyl, benzofluorenyl, phenanthryl, triphenyl enyl, pyrenyl, perylenyl, chrysenyl, naphtacenyl, fluoranthenyl and the like.

“Composition” and like terms refer to a mixture of two or more materials. Included in compositions are pre-reaction, reaction and post-reaction mixtures, the latter of which will include reaction products and by-products as well as unreacted components of the reaction mixture and decomposition products, if any, formed from the one or more components of the pre-reaction or reaction mixture.

The terms “comprising,” “including,” “having,” and their derivatives, are not intended to exclude the presence of any additional component, step or procedure, whether or not the same is specifically disclosed. In order to avoid any doubt, all compositions claimed through use of the term “comprising”may include any additional additive, adjuvant, or compound, whether polymeric or otherwise, unless stated to the contrary. In contrast, the term, “consisting essentially of” excludes from the scope of any succeeding recitation any other component, step, or procedure, excepting those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa.

A “cycloalkyl” is a saturated cyclic non-aromatic hydrocarbon radical having a single ring or multiple condensed rings. Suitable cycloalkyl radicals include, for example, cyclopentyl, cyclohexyl, cyclooctyl, bicyclooctyl, etc. In particular embodiments, cycloalkyls have between 3 and 200 carbon atoms, between 3 and 50 carbon atoms or between 3 and 20 carbon atoms.

A “heteroatom” is an atom other than carbon or hydrogen. The heteroatom can be a non-carbon atom from Groups IV, V, VI and VII of the Periodic Table. Nonlimiting examples of heteroatoms include: F, N, O, P, B, S, and Si.

“Hydrocarbyl” and “hydrocarbon” refer to substituents containing only hydrogen and carbon atoms, including branched or unbranched, saturated or unsaturated, cyclic, polycyclic or acyclic species, and combinations thereof. Nonlimiting examples of hydrocarbyl groups include alkyl-, cycloalkyl-, alkenyl-, alkadienyl-, cycloalkenyl-, cycloalkadienyl-, aryl-, aralkyl, alkylaryl, and alkynyl-groups.

“Substituted hydrocarbyl” and “substituted hydrocarbon” refer to a hydrocarbyl group that is substituted with one or more non-hydrocarbyl substituent groups. Nonlimiting examples of a non-hydrocarbyl substituent group include a heteroatom, heteroatom-containing moieties, oxygen-containing moieties (e.g., alcohol, acrylate, acrylic acid, aldehyde, carboxylic acid, ester, ether, ketone, and peroxide groups), and nitrogen-containing moieties (e.g., amide, amine, azo, imide, imine, nitrate, nitrile, and nitrite groups).

All reference to the Periodic Table of the Elements shall refer to the Periodic Table of the Elements, published and copyrighted by CRC Press, Inc., 2003. Also, any reference to a Group or Groups shall be to the Group or Groups as reflected in this Periodic Table of the Elements using the IUPAC system for numbering groups.

The numerical ranges disclosed herein include all values from, and including, the lower and upper value. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6; etc.).

As used herein, the term “percent weight” refers to the amount of a component relative to the entire weight of the composition, expressed as: (mass of the component/mass of the composition)×100%.

Unless stated to the contrary, implicit from the context, or customary in the art, all parts and percents are based on weight and all test methods are current as of the filing date of this disclosure. It is understood that the sum of the components in each of the mixtures and compositions, disclosed herein yields 100 weight percent.

For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. Skilled artisans will recognize the examples provided herein have many useful alternatives and fall within the scope of embodiments of the invention.

The present disclosure provides a composition. The composition contains (A) a psychoactive compound selected from psilocybin, psilocin, and combinations thereof; and (B) a supplement selected from an amino acid, a vitamin B6, piracetam, gamma aminobutyric acid, theobromine, caffeine, resveratrol, and combinations thereof.

A. Psychoactive Compound

The composition contains a psychoactive compound selected from psilocybin, psilocin, and combinations thereof. As used herein, a “psychoactive compound” is a chemical substance that is capable of changing the nervous system function of an individual, resulting in alterations to the individuals perception, mood, consciousness, cognition, behavior, or a combination thereof.

In an embodiment, the psychoactive compound is psilocybin. Psilocybin is also known as 4-phosphoryloxy-N,N-dimethyl-tryptamine or [3-(2-trimethylaminoethyl)-lH-indol-4-yl]dihydrogen phosphate). Psilocybin has the following Structure A:

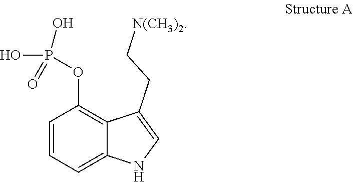 [SMILES: CN(C)CCc1c[nH]c2cccc(OP(=O)(O)O)c12]

Psilocybin has, for example, been used as an aide to psychotherapy for the treatment of mood disorders and alcoholic disorders, and for relieving symptoms of depression (Daniel et al., Ment Health Clin. 7(1):24-28 (2017); Griffiths et al., J Psychopharmacol 30 (12): 1181-1197 (2016); Ross et al., J Psychopharmacol 30 (12): 1165-1180 (2016); and Carhart-Harris et al., Lancet Psychiatry 3(7): 619-627 (2016)).

Psilocybin has also been found to increase global neural signal diversity (Schartner et al. Nature Scientific Reports, 7:46421 (2017)). Also, in a study examining the effects of psilocybin on hippocampal neurogenesis and extinction of trace fear conditioning, it was found that psilocybin facilitates extinction of the classically conditioned fear response and thus may have potential for treatment of post-traumatic stress disorder and related conditions (Catlow et al., Experimental Brain Research 228:481-491 (2013)).

In an embodiment, the psychoactive compound is psilocin. Psilocin is also known as 4-hydroxy-N,N-dimethyltryptamine. Psilocin has the following Structure B:

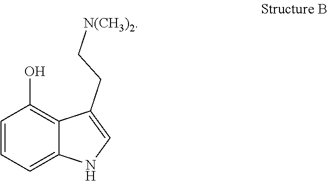 [SMILES: CN(C)CCc1c[nH]c2cccc(O)c12]

In an embodiment, the composition contains psilocybin and psilocin.

In an embodiment, the composition contains from about 10 wt % to about 30 wt % of the psychoactive compound, based on the total weight of the composition. In another embodiment, the composition contains from 10 wt %, or 12 wt %, or 15 wt %, or 16 wt %, or 18 wt %, or 20 wt % to 22 wt %, or 24 wt %, or 25 wt %, or 28 wt %, or 30 wt % of the psychoactive compound, based on the total weight of the composition. In a further embodiment, the composition contains from 10 wt % to 30 wt %, or from 12 wt % to 28 wt %, or from 15 wt % to 25 wt %, or from 16 wt % to 24 wt %, or from 18 wt % to 22 wt % or from 20 wt % to 25 wt %% of the psychoactive compound, based on the total weight of the composition.

The psychoactive compound may comprise two or more embodiments disclosed herein.

B. Supplement

The composition contains a supplement selected from an amino acid, a vitamin B6, piracetam, gamma aminobutyric acid, theobromine, caffeine, resveratrol, and combinations thereof.

In an embodiment, the supplement is an amino acid. An “amino acid” is an organic compound having the following Structure C:

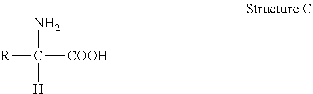

wherein R is selected from hydrogen, a hydrocarbon, and a substituted hydrocarbon.

In an embodiment, in Structure C, the R group is selected from hydrogen, a C1-C20 hydrocarbon, and a C1-C20 substituted hydrocarbon. In another embodiment, in Structure C, the R group is selected from hydrogen, a C1-C9 hydrocarbon, and a C1-C9 substituted hydrocarbon.

Nonlimiting examples of suitable amino acids include tryptophan, arginine, phenylalanine, tyrosine, histidine, arginine, alanine, glycine, serine, threonine, leucine, isoleucine, methionine, valine, theanine, and combinations thereof. In an embodiment, the amino acid is selected from L-tryptophan, L-arginine, L-phenylalanine, L-tyrosine, L-histidine, L-arginine, 5- hydroxytryptophan (5-HTTP), theanine, and combinations thereof.

In an embodiment, the amino acid is 5-hydroxytryptophan (5-HTTP). 5-HTTP is also known as oxitriptan.

In an embodiment, the amino acid is selected from L-tryptophan, L-arginine, L-phenylalanine, L-tyrosine, L-histidine, L-arginine, and combinations thereof.

In an embodiment, the supplement is vitamin B6. “Vitamin B6” is a B vitamin that functions as a coenzyme in enzymatic reactions in metabolism. Nonlimiting examples of vitamin B6 include pyridoxine (PN), pyridoxine 5′-phosphate (P5P), pyridoxal (PL), pyridoxal 5′-phosphate (PLP), pyridoxamine (PM), pyridoxamine 5′-phosphate (PMP), 4-pyroxidic acid (PA), pyritinol, and combinations thereof. In an embodiment, the vitamin B6 is pyridoxine.

In an embodiment, the supplement is piracetam. Piracetam is also known as 2-oxo-1-pyrrolidine acetamide.

In an embodiment, the supplement is gamma aminobutyric acid (GABA). GABA is also known as 4-aminobutanoic acid.

In an embodiment, the supplement is theobromine. Theobromine is also known as 3,7-dimethylxanthine.

In an embodiment, the supplement is caffeine. Caffeine is also known as 1,3,7-trimethylxanthine.

In an embodiment, the supplement is resveratrol. Resveratrol is also known as 3,5,4′- trihydroxystilbene.

In an embodiment, the supplement is selected from L-tryptophan, L-arginine, L-phenylalanine, L-tyrosine, L-histidine, L-arginine, 5-HTTP, pyridoxine, piracetam, GABA, theobromine, caffeine, resveratrol, and combinations thereof.

In an embodiment, the supplement is selected from piracetam, GABA, and combinations thereof.

In an embodiment, the supplement is selected from theobromine, caffeine, and combinations thereof.

In an embodiment, the supplement is selected from an amino acid and vitamin B6. In a further embodiment, the supplement is selected from L-tryptophan, L-arginine, L-phenylalanine, L-tyrosine, L-histidine, L-arginine, 5-HTTP, PN, PSP, PL, PLP, PM, PMP, PA, pyritinol, and combinations thereof. In a further embodiment, the supplement is selected from L-tryptophan, L-arginine, L-phenylalanine, L-tyrosine, L-histidine, L-arginine, 5-HTTP, PN, and combinations thereof.

In an embodiment, the supplement is selected from L-tryptophan, L-arginine, L-phenylalanine, 5-HTTP, vitamin B6, piracetam, theobromine, caffeine, GABA, resveratrol, and combinations thereof.

In an embodiment, the supplement is a composition containing, consisting essentially of, or consisting of L-tryptophan, L-arginine, L-phenylalanine, 5-HTTP, vitamin B6, piracetam, theobromine, caffeine, GABA, and resveratrol.

In some embodiments, the composition contains from 20 wt % to 90 wt %, or from 25 wt % to 75 wt %, or from 25 wt % to 50 wt %, or from 25 wt % to 35 wt % of the supplement, based on the total weight of the composition. In an embodiment, the composition contains from 20 wt %, or 25 wt %, or 30 wt % to 35 wt %, or 40 wt %, or 50 wt %, or 55 wt %, or 60 wt %, or 70 wt %, or 80 wt %, or 90 wt % of the supplement, based on the total weight of the composition. In another embodiment, the composition contains from about 25 wt % to about 35 wt % of the supplement, based on the total weight of the composition.

In an embodiment, the composition contains, based on the total weight of the composition:

(i) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-tryptophan; and/or

(ii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-arginine; and/or

(iii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % vitamin B6; and/or

(iv) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % piracetam; and/or

(v) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % theobromine; and/or

(vi) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % 5-HTTP; and/or

(vii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % GABA; and/or

(viii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % resveratrol; and/or (ix) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-phenylalanine; and/or

(x) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % caffeine; and/or

(xi) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % theanine.

e supplement may comprise two or more embodiments disclosed herein.

C. Optional Additive

In an embodiment, the composition contains (A) the psychoactive compound selected from psilocybin, psilocin, and combinations thereof; (B) the supplement selected from an amino acid, a vitamin B6, piracetam, gamma aminobutyric acid, theobromine, caffeine, resveratrol, and combinations thereof; and (C) one or more optional additive.

Nonlimiting examples of suitable additives include creatine, niacin, vitamin C, nattokinase, choline, thiamine, sulbutiamine, glutathione, agmatine, plant herb, flavorings (natural or artificial), colorants, and combinations thereof.

In an embodiment, the additive is selected from creatine, niacin, vitamin C, nattokinase, choline, thiamine, sulbutiamine, glutathione, agmatine, plant herb, and combinations thereof.

In an embodiment, the additive is selected from creatine, niacin, vitamin C, nattokinase, choline, thiamine, sulbutiamine, glutathione, agmatine, and combinations thereof.

In an embodiment, the additive is creatine. Creatine is also known as methylguanidoacetic acid.

In an embodiment, the additive is niacin. Niacin is also known as nicotinic acid.

In an embodiment, the additive is vitamin C. “Vitamin C” is an essential nutrient involved in the repair of tissue and the enzymatic production of certain neurotransmitters. Nonlimiting examples of suitable vitamin C include ascorbic acid, sodium ascorbate, and combinations thereof.

In an embodiment, the additive is nattokinase.

In an embodiment, the additive is choline. Choline is a water-soluble quaternary ammonium compound that is an essential nutrient. Nonlimiting examples of suitable choline include choline, choline bitartrate, choline phospholipids, lecithin, and combinations thereof. A nonlimiting example of a suitable choline phospholipid is phosphatidylcholine.

In an embodiment, the additive is thiamine. Thiamine is also known as aneurin.

In an embodiment, the additive is sulbutiamine. Sulbutiamine is a synthetic derivative of thiamine.

In an embodiment, the additive is glutathione.

In an embodiment, the additive is agmatine. Agmatine is also known as (4-aminobutyl)guanidine

In an embodiment, the additive is a plant herb. A “plant herb” is a medicinal preparation prepared from a plant or plant part. Nonlimiting examples of suitable plant herbs include rhodiola, ephedra, ashwagandha, ginseng, guarana, and combinations thereof. The plant herb may be in the form of a powder, an extract, a pulp, a leaf, a stem, a flower, a petal, a seed, a root, or a combination thereof.

In an embodiment, the plant herb is rhodiola. “Rhodiola” is a medicinal preparation from the plant Rhodiola rosea. In an embodiment, the rhodiola contains one or more of phenols, rosavin, rosin, rosarin, organic acids, terpenoids, phenolic acids and their derivatives, flavonoids, anthraquinones, alkaloids, tyrosol, and salidroside.

In an embodiment, the plant herb is ephedra. “Ephedra” is a medicinal preparation from the plant Ephedra sinica. In an embodiment, the ephedra contains one or more of ephedrine, pseudoephedrine (isoephedrine), norpseudoephedrine (cathine), norephedrine, methylephedrine, and methylpseudoephedrine.

In an embodiment, the plant herb is ashwagandha. “Ashwagandha” is a medicinal preparation from the plant Withania somnifera. In an embodiment, the ashwagandha contains one or more of sitoindoside, withanolide (withaferin A and/or withanone), acylsterylglucoside, triethylene glycol, alkaloids, and saponins.

In an embodiment, the plant herb is ginseng. “Ginseng” is a medicinal preparation from plants of the genus Panax, such as Panax ginseng, Panax notoginseng, Panax qidnquefolius, and combinations thereof. In an embodiment, the ginseng contains one or more of ginsenosides (e.g., Rgl, Rc, Rd, Re, Rb1, Rb2, RbO, etc.) and pseudoginsenosides.

In an embodiment, the plant herb is guarana. “Guarana” is a medicinal preparation from the plant Paulliiiia cupana. In an embodiment, the guarana contains one or more of caffeine, catechutannic-acid, choline, D-catechin, guanine, hypoxanthine, mucilage, resin, saponin, starch, tannin, theobromine, theophylline, timbonine, and xanthine.

In an embodiment, the composition contains from about 5 wt % to about 35 wt % additive, based on the total weight of the composition. In another embodiment, the composition contains from 5 wt %, or 10 wt %, or 20 wt %, or 25 wt % to 30 wt %, or 35 wt % additive, based on the total weight of the composition. In a further embodiment, the composition contains from 5 wt % to 35 wt %, or from 5 wt % to 10 wt %, or from 5 wt % to 25 wt %, or from 10 wt % to 20 wt %, or from 15 wt % to 25 wt %, or from 25 wt % to 35 wt %, or from 25 wt % to 30 wt %, or from 30 wt % to 35 wt % additive, based on the total weight of the composition.

In an embodiment, the composition contains from about 25 wt % to about 35 wt % of an additive selected from creatine, niacin, vitamin C, nattokinase, choline, thiamine, sulbutiamine, glutathione, agmatine, and combinations thereof, based on the total weight of the composition. In a further embodiment, the composition contains from 25 wt % to 35 wt %, or from 25 wt % to 30 wt %, or from 30 wt % to 35 wt % of an additive selected from creatine, niacin, vitamin C, nattokinase, choline, thiamine, sulbutiamine, glutathione, agmatine, and combinations thereof, based on the total weight of the composition.

In an embodiment, the composition contains from about 5 wt % to about 10 wt % of an additive that is a plant herb selected from rhodiola, ephedra, ashwagandha, ginseng, guarana, and combinations thereof, based on the total weight of the composition. In a further embodiment, the composition contains from 5 wt % to 10 wt %, or from 6 wt % to 10 wt %, or from 5 wt % to 9 wt % of an additive that is a plant herb selected from rhodiola, ephedra, ashwagandha, ginseng, guarana, and combinations thereof, based on the total weight of the composition.

In an embodiment, the composition contains (i) from 25 wt % to 35 wt %, or from 25 wt % to 30 wt %, or from 30 wt % to 35 wt % of an additive selected from creatine, niacin, vitamin C, nattokinase, choline, thiamine, sulbutiamine, glutathione, agmatine, and combinations thereof; and (ii) from 5 wt % to 10 wt %, or from 6 wt % to 10 wt %, or from 5 wt % to 9 wt % of an additive that is a plant herb selected from rhodiola, ephedra, ashwagandha, ginseng, guarana, and combinations thereof, based on the total weight of the composition.

The optional additive may comprise two or more embodiments disclosed herein. I). D. Composition

The composition contains (A) the psychoactive compound selected from psilocybin, psilocin, and combinations thereof; and (B) the supplement selected from an amino acid, a vitamin B6, piracetam, gamma aminobutyric acid, theobromine, caffeine, resveratrol, and combinations thereof.

In an embodiment, the composition contains (A) the psychoactive compound selected from psilocybin, psilocin, and combinations thereof; (B) the supplement selected from an amino acid, a vitamin B6, piracetam, gamma aminobutyric acid, theobromine, caffeine, resveratrol, and combinations thereof; and (C) one or more optional additive. In a further embodiment, the optional additive is selected from creatine, niacin, vitamin C, nattokinase, choline, thiamine, sulbutiamine, glutathione, agmatine, plant herb, flavorings, colorants, and combinations thereof

In an embodiment, the composition contains, consists essentially of, or consists of, based on the total weight of the composition:

(A) from 10 wt % to 30 wt %, or from 12 wt % to 28 wt %, or from 15 wt % to 25 wt %, or from 16 wt % to 24 wt %, or from 18 wt % to 22 wt % or from 20 wt % to 25 wt % of the psychoactive compound selected from psilocybin, psilocin, and combinations thereof;

(B) from 20 wt % to 90 wt %, or from 25 wt % to 75 wt %, or from 25 wt % to 50 wt %, or from 25 wt % to 35 wt % of the supplement selected from an amino acid, a vitamin B6, piracetam, gamma aminobutyric acid, theobromine, caffeine, resveratrol, and combinations thereof; and

(C) optionally, from 5 wt % to 35 wt %, or from 5 wt % to 10 wt %, or from 5 wt % to 25 wt %, or from 10 wt % to 20 wt %, or from 15 wt % to 25 wt %, or from 25 wt % to 35 wt %, or from 25 wt % to 30 wt %, or from 30 wt % to 35 wt % additive.)

In an embodiment, the composition contains, consists essentially of, or consists of, based on the total weight of the composition:

(A) from 10 wt % to 30 wt %, or from 12 wt % to 28 wt %, or from 15 wt % to 25 wt %, or from 16 wt % to 24 wt %, or from 18 wt % to 22 wt % or from 20 wt % to 25 wt %% of the psychoactive compound selected from psilocybin, psilocin, and combinations thereof;

(B) from 20 wt % to 90 wt %, or from 25 wt % to 75 wt %, or from 25 wt % to 50 wt %, or from 25 wt % to 35 wt % of the supplement selected from an amino acid, a vitamin B6, piracetam, gamma aminobutyric acid, theobromine, caffeine, resveratrol, and combinations thereof; and (C) optionally, from 25 wt % to 35 wt %, or from 25 wt % to 30 wt %, or from 30 wt % to 35 wt % of a first additive selected from creatine, niacin, vitamin C, nattokinase, choline, thiamine, sulbutiamine, glutathione, agmatine, and combinations thereof; and

(D) optionally, from 5 wt % to 10 wt %, or from 6 wt % to 10 wt %, or from 5 wt % to 9 wt % of a second additive that is a plant herb selected from rhodiola, ephedra, ashwagandha, ginseng, guarana, and combinations thereof.

In an embodiment, the composition contains, consists essentially of, or consists of, based on the total weight of the composition:

(A) from 10 wt % to 30 wt %, or from 12 wt % to 28 wt %, or from 15 wt % to 25 wt %, or from 16 wt % to 24 wt %, or from 18 wt % to 22 wt % or from 20 wt % to 25 wt %% of the psychoactive compound selected from psilocybin, psilocin, and combinations thereof;

(B) a supplement selected from one or more of:

(i) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-tryptophan; and/or

(ii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-arginine; and/or

(iii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % vitamin B6; and/or

(iv) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % piracetam; and/or

(v) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % theobromine; and/or

(vi) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % 5-HTTP; and/or (vii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % GABA; and/or

(viii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % resveratrol; and/or

(ix) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-phenylalanine; and/or

(x) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % caffeine; and/or

(xi) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % theanine; and

(C) optionally, from 5 wt % to 35 wt %, or from 5 wt % to 10 wt %, or from 5 wt % to 25 wt %, or from 10 wt % to 20 wt %, or from 15 wt % to 25 wt %, or from 25 wt % to 35 wt %, or from 25 wt % to 30 wt %, or from 30 wt % to 35 wt % additive; wherein the total amount of supplement (i.e., a combined amount of the one or more of (i) (xi)) in the composition is from 20 wt % to 90 wt %, or from 25 wt % to 75 wt %, or from 25 wt % to 50 wt %, or from 25 wt % to 35 wt %.

In an embodiment, the composition contains, consists essentially of, or consists of, based on the total weight of the composition:

(A) from 10 wt % to 30 wt %, or from 12 wt % to 28 wt %, or from 15 wt % to 25 wt %, or from 16 wt % to 24 wt %, or from 18 wt % to 22 wt % or from 20 wt % to 25 wt %% of the psychoactive compound selected from psilocybin, psilocin, and combinations thereof;

(B) a supplement selected from one or more of:

(i) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-tryptophan; and/or (ii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-arginine; and/or

(iii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % vitamin B6; and/or

(iv) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % piracetam; and/or

(v) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % theobromine; and/or

(vi) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % 5-HTTP; and/or

(vii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % GABA; and/or

(viii) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % resveratrol; and/or

(ix) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % L-phenylalanine; and/or

(x) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % caffeine; and/or

(xi) from 2 wt % to 10 wt %, or from 2 wt % to 7 wt %, or from 2 wt % to 5 wt %, or from 2 wt % to 3 wt %, or from 4 wt % to 10 wt %, or from 5 wt % to 10 wt %, or from 6 wt % to 10 wt %, or from 8 wt % to 10 wt % theanine; (C) optionally, from 25 wt % to 35 wt %, or from 25 wt % to 30 wt %, or from 30 wt % to 35 wt % of a first additive selected from creatine, niacin, vitamin C, nattokinase, choline, thiamine, sulbutiamine, glutathione, agmatine, and combinations thereof; and

(D) optionally, from 5 wt % to 10 wt %, or from 6 wt % to 10 wt %, or from 5 wt % to 9 wt % of a second additive that is a plant herb selected from rhodiola, ephedra, ashwagandha, ginseng, guarana, and combinations thereof; wherein the total amount of supplement (i.e., a combined amount of the one or more of (i)-(xi)) in the composition is from 20 wt % to 90 wt %, or from 25 wt % to 75 wt %, or from 25 wt % to 50 wt %, or from 25 wt % to 35 wt %.

The composition may be in the form of an oral dosage, such as a tablet, a capsule, or a suspension. In an embodiment, the oral dosage comprises (i) the composition and (ii) one or more of a pharmaceutically acceptable carrier, an excipient, and a diluent. It is understood that the weight percents provided for the above-listed compositions exclude the pharmaceutically acceptable carrier, excipient, and diluent.

Not wishing to be bound by any particular theory, it is believed that the composition containing (A) the psychoactive compound selected from psilocybin, psilocin, and combinations thereof; and (B) the supplement selected from an amino acid, a vitamin B6, piracetam, gamma aminobutyric acid, theobromine, caffeine, resveratrol, and combinations thereof may, following ingestion, advantageously provide an individual with one or more of antioxidant, anticancer, antidiabetic, antiallergic, immunomodulating, cardiovascular protector, anticholesterolemic, antiviral, antibacterial, antiparasitic, antifungal, detoxification, hepatoprotective, and anti inflammatory effects. Further, it is believed that the composition may, following ingestion, advantageously alleviate anxiety, ease depression, promote better sleep, promote healing, sharpen focus, increase concentration, reduce irritability, improve memory function, lower blood pressure, increase circulation, lower cholesterol, improve athletic performance, speed up muscle recovery, and/or increase energy for an individual.

The composition may comprise two or more embodiments disclosed herein.

Method for Making a Composition

The present disclosure provides a method for making a composition. The method includes (a) providing ethanol, distilled from a rye-containing mash; (b) providing dried and cured psilocybin-containing mushrooms, and grinding the mushrooms to obtain a particulate material; (c) soaking the particulate material in water; (d) adding distilled ethanol to the soaked particulate material, mixing to carry out ethanol extraction, and filtering to obtain an ethanol extract; (e) adding distilled ethanol to the ethanol extract to yield a solution; (f) stirring the solution; (g) heating the solution while stirring to cause evaporation, thereby obtaining a sludge; and (h) drying the sludge using vacuum to produce a powder material, thereby obtaining the composition.

a. Providing Ethanol

Ethanol is provided. The ethanol is distilled from a rye-containing mash. In other words, the ethanol is a bioethanol produced by preparing a rye-containing (i.e., Secale cereale-containing mash), fermenting the rye-containing mash, and distilling the fermented product to yield ethanol. Bioethanol excludes synthetic ethanol.

In an embodiment, step (a) comprises mixing powdered rye, wheat, and malt with sufficient water to produce a thick suspension (i.e., a rye-containing mash), covering the suspension with a porous material that allows air flow and inoculation by mold, and allowing fermentation to occur to obtain a fermented material. Water may be added to this fermented material and fermentation allowed to continue, followed by distilling to obtain distilled ethanol. In an embodiment, one or more sugars may be added to the powdered rye, wheat, and malt. Nonlimiting examples of suitable sugars include organic sugar, maple sugar, and combinations thereof.

In an embodiment, the rye-containing mash is prepared with water, rye grain, wheat grain, and dray extract of malt. The rye-containing mash is stirred and placed in a container (e.g., a glass or stainless-steel container) and covered with a material that enables air flow and inoculation by air borne mold (e.g., with a cheese-cloth type material). A nonlimiting example of suitable air borne mold is ergot mold spores. The mixture is left to ferment, and is periodically stirred (e.g., once a day). Water may be added to maintain a soup-like texture. Fermentation and mixing continue until, upon visual inspection, a consistent “fungal” infection is identified. Then, the fermenting material is left (without stirring) for at least 4 days (or from 4, or 5 to 10, or 12, or 20 days), until the mixture becomes a solid. Water may be added to the fermented material (e.g., an additional 0.25 to 0.5 gallons of water), and the fermented material left to sit and further ferment for at least 4 days (or from 4, or 7 to 10, or 12, or 24 days). Ethanol is then distilled from the fermented material. Distillation may be performed using a stainless-steel thumper-style distillation arrangement, or by a laboratory apparatus made of glass.

In the methods described herein, fermentation may be carried out for from about 5 days to about 15 days, or from about 6 days to about 12 days. In an embodiment, fermentation is carried out for from 5, or 6, or 8, or 9, or 10 to 12, or 13, or 14, or 15 days. In a further embodiment, fermentation is carried out for from 5 days to 15 days, or from 6 days to 12 days, or from 5 days to 10 days.

b. Providing Psilocybin-Containing Mushrooms

Dried and cured psilocybin-containing mushrooms are provided. A “psilocybin-containing mushroom” is a mushroom that contains a psychoactive compound selected from psilocybin, psilocin, and combinations thereof. Nonlimiting examples of suitable psilocybin-containing mushrooms in the genus Conocybe, Copelandia, Calerina, Gymnopilus, Inocybe, Panaeolus, Philiotina, Pluteus, and Psilocybe.

In an embodiment, the psilocybin-containing mushrooms are of the genus Psilocybe. Nonlimiting examples of suitable psilocybin-containing mushrooms that are in the genus Psilocybe include Psilocybe atlantis, Psilocybe cubensis, Psilocybe tampanensis, Psilocybe Mexicana, Psilocybe ovoideocystidiata, Psilocybe semilanceata, Psilocybe weraroa, Psilocybe zapotacorum, Psilocybe yungensis, Psilocybe xalapensis, Psilocybe venenata, Psilocybe subtropicalis, Psilocybe singer, Psilocybe schultesii, Psilocybe rostrata, Psilocybe quebecensis, Psilocybe pintonii, Psilocybe puberula, Psilocybe mairei, Psilocybe laurae, Psilocybe kumaenorum, Psilocybe heimii, Psilocybe galindoi, Psilocybe fmetaria, Psilocybe egonii, Psilocybe dumontii, Psilocybe carbonaria, Psilocybe cordispora, Psilocybe bispora, Psilocybe aucklandii, and combinations thereof.

The psilocybin-containing mushrooms are dried and cured. Nonlimiting examples of suitable methods of drying and curing the psilocybin-containing mushrooms include heating, leaving exposed to the atmosphere for a period of time (e.g., from 1 to 30 days), storing in the presence of a desiccant (i.e., a substance that absorbs water from the atmosphere) for a period of time (e.g., from 1 to 30 days), and/or placing in a dehydrator for a period of time (e.g., from 1 to 24 hours).

The dried and cured psilocybin-containing mushrooms are ground to obtain a particulate material. Nonlimiting examples of suitable grinding methods include mortar-and-pestle, blending, grist milling, and combinations thereof.

In an embodiment, the particulate material is a mixture of powder and particulates, such as mixture of about 50 wt % powder and about 50 wt % particulate, based on the total weight of the particulate material.

c. Soaking the Particulate Material in Water

The particulate material is soaked in water. In an embodiment, the particulate material is soaked in water for at least 3 days, or at least 4 days, or at least 5 days, or at least 6 days, or at least 7 days, or at least 8 days. In an embodiment, the particulate material is soaked in water for from 3 days to 20 days, or from 3 days to 12 days, or from 3 days to 10 days, or from 4 days to 10 days, or from 4 days to 6 days, or from 4 days to 5 days. In an embodiment, the water is distilled water. A soaked particulate material is formed.

d. Adding Distilled Ethanol to the Soaked Particulate Material

Distilled ethanol is added to the soaked particulate material. In an embodiment, the distilled ethanol is the ethanol distilled from rye-containing mash.

After adding the distilled ethanol to the soaked particulate material, the components are mixed to carry out ethanol extraction and filtered to obtain an ethanol extract.

In an embodiment, a mixture containing the distilled ethanol and the soaked particulate material is mixed for at least 8 hours, or at least 9 hours, or at least 10 hours, or at least 11 hours, or at least 12 hours. In another embodiment, the mixture containing the distilled ethanol and the soaked particulate material is mixed for from 8 hours to 24 hours, or from 8 hours to 18 hours, or from 8 hours to 14 hours, or from 10 hours to 14 hours. Nonlimiting examples of suitable mixing methods include blending and stirring.

The mixture is filtered to obtain an ethanol extract. A nonlimiting example of a suitable filtering method includes pouring the mixture over fine cheese cloth. In an embodiment, when pouring the mixture containing the distilled ethanol and the soaked particulate material over fine cheese cloth, a residue is caught in the cheese cloth and a filtrate passes through the cheese cloth and is collected.

In an embodiment, the residue caught in the cheese cloth is rinsed at least once, or at least twice with distilled ethanol and the ethanol rinse that passes through the cheese cloth is collected. In an embodiment, the residue caught in the cheese cloth is rinsed from 1, or 2, or 3 to 6, or 10, or 12 times with distilled ethanol. In an embodiment, the distilled ethanol is the ethanol distilled from rye-containing mash.

The filtrate that passed through the cheese cloth is combined with the ethanol rinse that passed through the cheese cloth to form the ethanol extract. e. Adding Distilled Ethanol to the Ethanol Extract

Distilled ethanol is added to the ethanol extract to yield a solution. In an embodiment, the distilled ethanol is the ethanol distilled from rye-containing mash.

In an embodiment, the solution contains about 75 volume percent (vol %) ethanol extract and about 25 vol % distilled ethanol, based on the total volume of the solution. In an embodiment, the solution contains from 50 vol %, or 60 vol %, or 70 vol %, or 75 wt % to 80 vol %, or 85 vol %, or 90 vol % ethanol extract; and a reciprocal amount of distilled ethanol, or from 10 vol %, or 15 vol %, or 20 vol % to 25 vol %, or 30 vol %, or 40 vol %, or 50 vol % distilled ethanol, based on the total volume of the solution.

In an embodiment, an amount of distilled ethanol is added to the ethanol extract that is sufficient to obtain a solution that may be stirred.

f. Stirring the Solution

The solution is stirred. In an embodiment, the solution is stirred with a magnetic stirrer, or further a reversible heated magnetic stirrer.

In an embodiment, the solution is stirred for a period of at least 12 hours. In another embodiment, the solution is stirred for a period of from 12 hours to 14 hours, or 18 hours, or 24 hours. In another embodiment, the solution is stirred for at least 1 day, or at least 2 days, or at least 3 days, or at least 4 days, or at least 5 days, or at least 6 days, or at least 7 days. In a further embodiment, the solution is stirred for a period of from 1 day, or 2 days, or 3 days, or 4 days, or 5 days to 6 days, or 7 days or 10 days, or 12 days.

In an embodiment, the solution is stirred without heat. In other words, the solution is stirred under ambient conditions (i.e., 15-25° C. (59-77° F.), 1 atm). In another embodiment, the solution is stirred at a temperature of no more than 5° F. greater than ambient temperature. In some embodiments, heat is applied using a heated reversible magnetic stirrer.

In an embodiment, the solution is stirred with a magnetic stirrer in a first direction for a period of from 12 hours to 24 hours, and is then stirred with the magnetic stirrer in a second (opposite) direction for a period of from 12 hours to 24 hours. This process is repeated for from 2 days, or 5 days to 7 days, or 10 days. In an embodiment, the stirring is continued until the solution is clear, or substantially clear.

In some embodiments, a supplement is provided. The supplement may be any supplement disclosed herein. In an embodiment, the supplement is provided in a powder or liquid form. Two or more supplements may be mixed together to form a supplement mixture before being added to the mixed solution. In another embodiment, one or more supplements are added directly to the mixed solution. In an embodiment, a supplement or supplement mixture is subjected to ultrasonic stimulation and stirring. The ultrasonic stimulation may be carried out in from 100, or 120, or 150, or 160 to 170, or 180, or 190, or 200 second intervals over at least 2 hours, or at least 3 hours, or at least 4 hours, or at least 6 hours. In some embodiments, the stimulated material is placed in a mixing device (e.g., a tumbler mixing device) with one or more types of mineral crystals (e.g., quartz, amethyst, magnetite, argonite, calcite, citrine, emerald, lapis lazuli, and combinations thereof) for mixing and/or grinding. The actual size of the mineral crystals is based on desired outcome of the final product. However, the size of the crystals is larger than size of any particles contained in the supplement. The supplement or supplement mixture is mixed and/or ground with the mineral crystals for at least 24 hours, or at least 48 hours. In an embodiment, the supplement or supplement mixture is mixed and/or ground with the mineral crystals for from 24 hours 120 hours, or from 48 hours to 96 hours. Then, the mineral crystals are separated from the supplement or supplement mixture. Without wishing to be bound by any particular theory, it is believed that mixing and/or grinding with mineral crystals enhances the direct and/or shared molecular bonding one all components are suspended in the ethanol solution. After being separated from the mineral crystals, the stimulated supplement or supplement mixture is added to the mixed solution and the combination is stirred. After the supplement or supplement mixture is added to the mixed solution, the combination is stirred. In an embodiment, stirring continues until the supplement or supplement mixture is dissolved, or substantially dissolved, in the ethanol of the solution.

In some embodiments, one or more optional additive is provided. The additive may be any additive disclosed herein. In an embodiment, the additive is provided in a powder or liquid form. The one or more additives may be mixed together to form an additive mixture before being added to the mixed solution. In some embodiments, the additive or additive mixture is placed in a mixing device (e.g., a tumbler mixing device) with one or more types of mineral crystals (e.g., quartz, amethyst, magnetite, argonite, calcite, citrine, emerald, lapis lazuli, and combinations thereof) for mixing and/or grinding. The actual size of the mineral crystals is based on desired outcome of the final product. However, the size of the crystals is larger than size of any particles contained in the additive or additive mixture. The additive or additive mixture is mixed and/or ground with the mineral crystals for at least 24 hours, or at least 48 hours. In an embodiment, the additive or additive mixture is mixed and/or ground with the mineral crystals for from 24 hours 120 hours, or from 48 hours to 96 hours. Then, the mineral crystals are separated from the additive or additive mixture. Without wishing to be bound by any particular theory, it is believed that mixing and/or grinding with mineral crystals enhances the direct and/or shared molecular bonding one all components are suspended in the ethanol solution. After being separated from the mineral crystals, the additive or additive mixture is added to the mixed solution and the combination is stirred. In another embodiment, the one or more additives is added directly to the mixed solution. The additive or additive mixture is added to the mixed solution and the combination is stirred.

In some embodiments, the one or more supplements may be pre-mixed with the one or more optional additives before the combination is added to the mixed solution. In another embodiment, the one or more supplements is added to the mixed solution separate from the one or more optional additives. The one or more supplements may be added before or after the one or more optional additives is added to the mixed solution. In some embodiments, the one or more supplements is added to the mixed solution first, the combination is mixed until the one more supplements is dissolved or substantially dissolved, then the one or more optional additives is added second, and the combination is mixed until the one or more additives is dissolved or substantially dissolved.

It is understood that the one or more supplements and/or the one or more optional additives may be added to the mixed solution during stirring or not. In an embodiment, the one or more supplements and/or the one or more optional additives is added to the mixed solution during stirring.

g. Heating the Solution

The solution is heated while being stirred to cause evaporation, thereby obtaining a sludge.

In some embodiments, after the supplement or supplement mixture is dissolved, or substantially dissolved, in the ethanol of the solution, heat is applied and stirring continues at a temperature of from 110° F., or 115° F., or 120° F. to 125° F., or 130° F., or 140° F., or 150° F. In some embodiments, the temperature while stirring is from 110° F. to 150° F., or from 110° F. to 140° F., or from 115° F. to 150° F., or from 120° F. to 150° F., or from 120° F. to 140° F., or from 120° F. to 130° F. While stirring under heat, the ethanol evaporates, leaving a concentrated sludge.

In some embodiments, after the additive or additive mixture is dissolved, or substantially dissolved, in the ethanol of the solution, heat is applied and stirring continues at a temperature of from 110° F., or 115° F., or 120° F. to 125° F., or 130° F., or 140° F., or 150° F. In some embodiments, the temperature while stirring is from 110° F. to 150° F., or from 110° F. to 140° F., or from 115° F. to 150° F., or from 120° F. to 150° F., or from 120° F. to 140° F., or from 120° F. to 130° F. While stirring under heat, the ethanol evaporates, leaving a concentrated sludge.

In some embodiments, the evaporated ethanol is captured and recycled.

h. Drying the Sludge

The sludge is dried using a vacuum to produce a powder material, thereby obtaining the composition.

In an embodiment, the sludge is placed in a vacuum purging device to evaporate any remaining ethanol and dry the sludge. The dried product is a powder or powder- like material. The dried product comprises the composition. The composition may be any composition disclosed herein.

In an embodiment, the powder material, or the composition, contains, consists essentially of, or consists of, based on the total weight of the composition:

(A) from 10 wt % to 30 wt %, or from 12 wt % to 28 wt %, or from 15 wt % to 25 wt %, or from 16 wt % to 24 wt %, or from 18 wt % to 22 wt % or from 20 wt % to 25 wt % of the psychoactive compound selected from psilocybin, psilocin, and combinations thereof;

(B) from 20 wt % to 90 wt %, or from 25 wt % to 75 wt %, or from 25 wt % to 50 wt %, or from 25 wt % to 35 wt % of the supplement selected from an amino acid, a vitamin B6, piracetam, gamma aminobutyric acid, theobromine, caffeine, resveratrol, and combinations thereof; and

(C) optionally, from 5 wt % to 35 wt %, or from 5 wt % to 10 wt %, or from 5 wt % to 25 wt %, or from 10 wt % to 20 wt %, or from 15 wt % to 25 wt %, or from 25 wt % to 35 wt %, or from 25 wt % to 30 wt %, or from 30 wt % to 35 wt % additive.

i. Method

The method includes (a) providing ethanol, distilled from a rye-containing mash; (b) providing dried and cured psilocybin-containing mushrooms, and grinding the mushrooms to obtain a particulate material; (c) soaking the particulate material in water; (d) adding distilled ethanol to the soaked particulate material, mixing to carry out ethanol extraction, and filtering to obtain an ethanol extract; (e) adding distilled ethanol to the ethanol extract to yield a solution; (f) stirring the solution; (g) heating the solution while stirring to cause evaporation, thereby obtaining a sludge; and (h) drying the sludge using vacuum to produce a powder material, thereby obtaining the composition.

In an embodiment, the method further includes providing a supplement selected from an amino acid, a vitamin B6, piracetam, gamma aminobutyric acid, theobromine, caffeine, resveratrol, and combinations thereof; and adding the supplement to the solution during stirring (i.e., in the step (f) disclosed above). Before being added to the solution, the supplement may be mixed with one or more mineral crystals and then separated from the mineral crystals.

In an embodiment, the method further includes providing an additive selected from creatine, niacin, vitamin C, nattokinase, choline, thiamine, sulbutiamine, glutathione, agmatine, plant herb, flavorings, colorants, and combinations thereof; and adding the additive to the solution during stirring (i.e., in the step (f) disclosed above). Before being added to the solution, the additive may be mixed with one or more mineral crystals and then separated from the mineral crystals.

In an embodiment, the method further includes forming an oral dosage comprising the composition. The oral dosage may be in the form of a tablet, a capsule, or a suspension. In an embodiment, the oral dosage comprises, consists essentially of, or consists of (i) the composition and (ii) one or more of a pharmaceutically acceptable carrier, an excipient, and a diluent.

The present disclosure also provides a composition, and further an oral dosage, produced by the above-described methods.

Not wishing to be bound by any particular theory, it is believed that the present composition may be useful in enhancing cognitive, emotional, and perceptual functions in a subject. Furthermore, the compositions described herein may be useful in promoting neurogenesis, resolving neuropathy, and improving neurological health. Such methods are encompassed herein.

Thus, in one aspect, the disclosure provides for methods of enhancing cognitive, emotional and perceptual functions by administering any of the compositions described herein to an individual in need thereof.

In another aspect, the disclosure provides for methods of treating a psychological disorder The psychological disorder may, for example, be an anxiety disorder, a depressive disorder, or a compulsive disorder. The anxiety disorder may be an acute stress disorder, anxiety due to a medical condition, generalized anxiety disorder, panic disorder, panic attack, a phobia, post-traumatic stress disorder, separation anxiety disorder, social anxiety disorder, substance-induced anxiety disorder, or selective mutism.

In other aspects, the disclosure provides a method of promoting neurogenesis, and/or resolving neuropathy, and/or improving neurological health by, administering any of the compositions described herein to an individual in need thereof.

The description is only exemplary of the principles of the disclosure, and should not be viewed as narrowing the scope of the claims that follow which claims define the full scope of the invention. Various aspects discussed in one drawing may be present and/or used in conjunction with the embodiment shown in another drawing, and each element shown in multiple drawings may be discussed only once. The described features, structures, or characteristics of configurations of the disclosure may be combined in any suitable manner in one or more configurations. In some cases, detailed description of well-known items or repeated description of substantially the same configurations may be omitted to facilitate the understanding of those skilled in the art by avoiding an unnecessarily redundant description. All statements herein reciting principles, aspects, and embodiments of the invention, as well as specific examples thereof, are intended to encompass equivalents thereof.

Reference in the specification to “one configuration” “one embodiment.” “a configuration” “an example.” or “an embodiment” means that a particular feature, structure, or characteristic described in connection with the configuration is included in at least one configuration, but is not a requirement that such feature, structure or characteristic be present in any particular configuration unless expressly set forth in the claims as being present. The appearances of the phrase “in one configuration” or “in one example” in various places may not necessarily limit the inclusion of a particular element of the disclosure to a single configuration, rather the element may be included in other or all configurations discussed herein.

As used in this specification and the appended claims, singular forms such as “a,” “an,” and “the” may include the plural unless the context clearly dictates otherwise. Thus, for example, reference to “a circular groove” may include one or more of such circular grooves, and reference to “the mouthpiece” may include reference to one or more of such mouthpieces.

As used herein, the term “generally” refers to something that is more of the designated adjective than not or the converse if used in the negative. As used herein, the term “about” is used to provide flexibility to a numerical range endpoint by providing that a given value may be “a little above” or “a little below” the endpoint while still accomplishing the function associated with the range, for example, “about” may be within 10% of the given number or given range. As used herein, a plurality of items, structural elements, compositional elements, and/or materials may be presented in a common list for convenience. However, these lists should be construed as though each member of the list is individually identified as a separate and unique member.

While methods are described herein in discrete steps in a particular order for the sake of clarity, the steps do not require a particular order and more than one step may be performed at the same time. For example, a later step may begin before earlier step completes Or, a later step may be completed before an earlier step is started. Additionally, the word “connected” and “coupled” is used throughout for clarity of the description and can include either a direct connection or an indirect connection.

Although the foregoing disclosure provides many specifics, such as use of the composition to treat certain diseases, symptoms, and disorders, it will be appreciated that other implementations are contemplated and these should not be construed as limiting the scope of any of the ensuing claims. Other embodiments and configurations may be devised which do not depart from the scopes of the claims. Features from different embodiments and configurations may be employed separately or in combination. Accordingly, all additions, deletions and modifications to the disclosed subject matter that fail within the scopes of the claims are to be and the full scope of available legal equivalents to its elements.

Furthermore, if any references have been made to patents and printed publications throughout this disclosure, each of these references and printed publications are individually incorporated herein by reference in their entirety.

1. A composition comprising:
(A) a psychoactive compound selected from the group consisting of psilocybin, psilocin, and combinations thereof; and
(B) a supplement selected from the group consisting of an amino acid, a vitamin B6, piracetam, gamma aminobutyric acid (GABA), theobromine, caffeine, resveratrol, and combinations thereof.

2. The composition of claim 1, wherein the composition comprises from 15 wt % to 25 wt % of the psychoactive compound, based on a total weight of the composition.

3. The composition of claim 2, wherein the supplement is an amino acid selected from the group consisting of L-tryptophan, L-arginine, L-phenylalanine, L-tyrosine, L-histidine, L-arginine, 5-hydroxy tryptophan (5-HTTP), theanine, and combinations thereof.

4. The composition of claim 3, further comprising an additive selected from the group consisting of creatine, niacin, vitamin C, nattokinase, choline, thiamine, sulbutiamine, glutathione, agmatine, and combinations thereof.

5. The composition of claim 4, further comprising a plant herb.

6. The composition of claim 5, wherein the plant herb is selected from the group consisting of rhodiola, ephedra, ashwagandha, ginseng, guarana, and combinations thereof.

7. The composition of claim 6, wherein the supplement comprises at least two of the amino acid, vitamin B6, piracetam, theobromine, GABA, reservatrol, and caffeine.

8. The composition of claim 7, wherein the additive comprises at least two of creatine, niacin, vitamin C, nattokinase, choline, thiamine, sulbutiamine, glutathione, and agmatine.

9. A method for making a composition comprising:
(a) providing ethanol, distilled from a rye containing mash;
(b) providing dried and cured psilocybin containing mushrooms, and grinding the mushrooms to obtain a particulate material;
(c) soaking the particulate material in water to form soaked particulate material;
(d) adding distilled ethanol to the soaked particulate material, mixing to carry out ethanol extraction, and filtering to obtain an ethanol extract;
(e) adding distilled ethanol to the ethanol extract to yield a solution;
(f) stirring the solution;
(g) heating the solution while stirring to cause evaporation, thereby obtaining a sludge; and
(h) drying the sludge using vacuum to produce a powder material, thereby obtaining the composition.

10. The method of claim 9, wherein step (a) comprises:
(i) mixing powdered rye, wheat, and malt with sufficient water to produce a suspension, covering the suspension with a porous material that allows air flow and inoculation by mold, and allowing fermentation to occur, thereby obtaining a fermented material;
(ii) adding water to the fermented material and allowing fermentation to continue; and
(iii) distilling the fermented material with ethanol, thereby obtaining distilled ethanol.

11. The method of claim 10, further comprising adding one or more sugars to the powdered rye, wheat, and malt.

12. The method of claim 10, wherein the fermentation is carried out for from 5 days to 15 days.

13. The method of claim 9, wherein the soaking is performed for at least three days.

14. The method of claim 9, wherein the mixing in step (d) is carried out for at least eight hours.

15. The method of claim 9, wherein the particulate material is about 50 wt % powder and about 50 wt % particulate, based on a total weight of the particulate material.

16. The method of claim 9, further comprising adding a supplement selected from the group consisting of an amino acid, a vitamin B6, piracetam, gamma aminobutyric acid (GABA), theobromine, caffeine, resveratrol, and combinations thereof, to the solution in step (f).

17. The method of claim 9, further comprising adding an additive selected from the group consisting of creatine, niacin, vitamin C, nattokinase, choline, thiamine, sulbutiamine, glutathione, agmatine, and combinations thereof, to the solution in step (f).

18. The method of claim 16, further comprising mixing the supplement with one or more mineral crystals and then separating the supplement from the mineral crystals before the supplement is added to the solution.

19. The method of claim 17, further comprising mixing additive with one or more mineral crystals and then separating the additive from the mineral crystals before the additive is added to the solution.

20. A method for making a composition comprising:
(a) providing ethanol;
(b) providing dried and cured psilocybin containing mushrooms, and grinding the mushrooms to obtain a particulate material;
(c) soaking the particulate material in water to form soaked particulate material;
(d) adding the ethanol to the soaked particulate material, mixing to carry out ethanol extraction, and filtering to obtain an ethanol extract;
(e) adding distilled ethanol to the ethanol extract to yield a solution;
(f) stirring the solution;
(g) heating the solution while stirring to cause evaporation, thereby obtaining a sludge; and
(h) drying the sludge to produce a powder material, thereby obtaining the composition.
